(19) United States
(12) Patent Application Publication
(10) Pub. No.: US 2003/0124549 A1
(43) Pub. Date: Jul. 3, 2003

(54) Devices and methods for detecting genetic sequences

(75) Inventors: Emest Beutler, La Jolla, CA (US); Richard Bruce, Los Altos, CA (US); Scott A. Elrod, La Honda, CA (US); John Stuart Fitch, Los Altos, CA (US); Huangpin Ben Hsieh, Palo Alto, CA (US); Eric Peeters, Fremont, CA (US); Richard A. Lerner, La Jolla, CA (US)

Correspondence Address: Bart W. Wise, 1300 I St. NW, Washington, DC, 20005-3315, US

(73) Assignees: Xerox Corporation; The Scripps Research Institute

(21) Appl. No.: 10/121,264

(22) Filed: Apr. 12, 2002

Related U.S. Application Data
(60) Provisional application No. 60/329,277, filed on Oct. 11, 2001.

(51) Int. Cl. C12Q 1/68; C12M 1/34

(52) U.S. Cl. 435/6; 435/287.2

(57) ABSTRACT

The invention provides devices for analyzing genetic material comprising a substrate and a first genetic material position and a second genetic material position on the substrate that each comprise genetic material attached to the substrate. The invention also provides methods of distributing genetic material comprising distributing at least one device.

The present application claims priority to or of U.S. Provisional Application Ser. No. 60/329,277, filed on Oct. 11, 2001, which is hereby incorporated by reference herein for any purpose.

### FIELD OF THE INVENTION

The invention relates to methods and materials for analyzing genetic material on substrates. Certain methods related to the packaging of such materials are also provided.

### BACKGROUND OF THE INVENTION

It is often desirable to analyze genetic material from more than one individual. In certain applications, it is useful to analyze genetic material from more than one individual of a population in order to determine the frequency of a particular genetic trait within such a population. In certain applications, it is useful to determine whether a particular genetic trait correlates with a particular phenotype of an individual. For example, it is often useful to determine whether a particular mutation, allele, or polymorphism correlates to a particular physiological condition, e.g., a disease condition. It may be useful to determine the frequency of a particular mutation, allele, or polymorphism in a given population.

### SUMMARY OF THE INVENTION

According to certain embodiments, devices are provided that comprise a substrate and a first genetic material position and a second genetic material position on the substrate that each comprise genetic material attached to the substrate. In certain embodiments, the genetic material of the first genetic material position comprises purified genomic DNA comprising more than one chromosome of the genomic DNA from at least one cell of an individual within a defined population and the genetic material of the second genetic material position comprises purified genomic DNA comprising more than one chromosome of the genomic DNA from at least one cell of an individual within a defined population. In certain embodiments, the genetic material of the first genetic material position is separate from the genetic material of the second genetic material position.

In certain embodiments, the device further comprises additional genetic material positions on the substrate that each comprise genetic material attached to the substrate. In certain embodiments, the genetic material of each of a majority of the additional genetic material positions are separate from one another. In certain embodiments, the genetic material of each of a majority of the genetic material positions derives from a different individual of the defined population.

According to certain embodiments, methods are provided for distributing genetic material. In certain embodiments, the methods comprise distributing at least one device to at least one recipient. In certain embodiments, the device comprises a substrate and a first genetic material position and a second genetic material position on the substrate that each comprise genetic material deposited onto the substrate. In certain embodiments, the genetic material of the first genetic material position comprises purified genomic DNA comprising more than one chromosome of the genomic DNA from at least one cell of an individual within a defined population and the genetic material of the second genetic material position comprises purified genomic DNA comprising more than one chromosome of the genomic DNA from at least one cell of an individual within a defined population. In certain embodiments, the substrate comprises a multiwell plate. In certain embodiments, the multiwell plate is one of a 96-well plate and a 384-well plate. In certain embodiments, each of a majority of the genetic material positions on the substrate comprises a solution comprising the genetic material.

In certain embodiments, the at least one device is distributed with information regarding the genetic material contained on the substrate. In certain embodiments, the information comprises genotyping information correlated with at least one position of the deposited genetic material.

According to certain embodiments, devices are provided that comprise a substrate comprising a matrix, one or more separate transfer agent spaces that extend through or partially through the matrix, and one or more transfer agent layers contained in one or more of the transfer agent spaces; and genetic material deposited on one or more of the transfer agent layers.

In certain embodiments, at least one of the transfer agent layers comprises a saccharide selected from at least one of sucrose and glucose. In certain embodiments, the transfer agent layers are contained in at least 96 separate transfer agent spaces. In certain embodiments, each of a majority of the at least 96 transfer agent spaces is alignable with a well of a 96-well plate. In certain embodiments, the genetic material on each of a majority of the transfer agent layers comprises purified genomic DNA comprising more than one chromosome of the genomic DNA from at least one cell of an individual within a defined population.

According to certain embodiments, methods are provided for distributing genetic material. In certain embodiments, the methods comprise distributing at least one device comprising a substrate comprising a matrix; one or more separate transfer agent spaces that extend through or partially through the matrix; one or more transfer agent layers contained in one or more of the transfer agent spaces. In certain embodiments, these methods comprise distributing at least one device to at least one recipient.

In certain embodiments, the at least one device is distributed with information regarding the genetic material contained on the substrate. In certain embodiments, the information comprises genotyping information correlated with at least one position of the deposited genetic material.

### BRIEF DESCRIPTION OF THE FIGURES

FIG. 1—FIG. 1 provides an illustration of certain embodiments that comprise a cover (80) with protrusions (40), wherein the protrusions comprise a capillary opening that extends into the protrusion. In certain embodiments, the capillary opening contains genetic material (50). In the embodiment shown, the cover is applied to a multiwell plate (20). In these embodiments, the wells of the multiwell plate contain a reaction volume comprising PCR primers and reagents (30). In these embodiments, insertion of the cover onto the plate will immerse the protrusions of the cover into the reaction volumes of the wells.

FIG. 2—FIG. 2A provides an illustration of certain embodiments that comprise a cover with lids (70) that align with the wells (60) of a multiwell plate. In these embodiments, genetic material (50) is attached on the cover within each lid of the cover. In these embodiments, the wells of the multiwell plate contain a reaction volume comprising PCR primers and reagents (30). FIG. 2B depicts certain embodiments that comprise a cover with genetic material (50) attached at genetic material positions that align with the wells of a multiwell plate (20). In the embodiments shown, each genetic material position is bordered by a sealing element (72) that is attached to the cover.

FIG. 3—FIG. 3 depicts a 10% polyacrylamide gel of PCR products amplified from genomic DNA ejected into vials from a piezo-actuated ejector as described in Example 2. Lanes 1-8 represent the amplified PCR products from samples ejected by the piezo-actuated ejector with increasing number of drops of genomic DNA solution deposited per vial from left to right. Lanes 9-16 represent the amplified PCR products from 8 control genomic DNA samples that had not been ejected by a piezo-actuated ejector. The two lanes (90) to the left of the lane with a DNA size marker (100) are negative controls consisting of water with no DNA.

FIG. 4—FIG. 4 provides a detailed perspective of certain embodiments of a substrate (110) that comprises a matrix and 96 transfer agent spaces (e.g. 112) that extend through the entire matrix and are alignable to the wells of a 96-well plate.

FIG. 5—FIG. 5 depicts certain embodiments of a substrate (110) and barrier layer (120) aligned to a 96-well plate (130), wherein the substrate comprises 96 transfer agent spaces containing transfer agent layers (e.g. 122) that extend into a matrix and that align with the wells of the 96-well plate. The perspective shown in FIG. 6 is depicted by the dotted line arrows labeled with the numeral 6.

FIG. 6—FIG. 6 depicts an end-on perspective of the certain embodiments described in FIG. 5.

FIG. 7 and FIG. 8—FIG. 7 and 8 illustrate certain embodiments of a process of transferring transfer agent layers (150) from 384 transfer agent holes that extend through an entire matrix (110) to multiple 96-well multiwell plates. In these embodiments, one transfer agent layer from one transfer agent hole (e.g. 140A) from each quadrant of four transfer agent holes (e.g. 140A, 140B, 140C, and 140D) containing transfer agent layers is transferred to a well of a 96-well plate (130). FIG. 7 represents the transfer of a first transfer agent layer from each quadrant to a first 96-well plate (130) and FIG. 8 represents the transfer of a second transfer agent layer from each quadrant to a second 96-well plate (132).

FIG. 9—FIG. 9 illustrates certain embodiments of transfer agent layers. A: Certain embodiments of a matrix (110) and non-tapered transfer agent holes (151) extending through the entire matrix, wherein the transfer agent holes contain transfer agent layers (150); B: Certain embodiments of a matrix (110) and tapered transfer agent holes (154) extending through the entire matrix, wherein the transfer agent holes contain transfer agent layers (150); C: Certain embodiments of a matrix (110) and tapered transfer agent indentations (152) extending partially through the entire matrix, wherein the transfer agent indentations contain transfer agent layers (150); D: Certain embodiments of a matrix (110) and tapered transfer agent indentations (152) extending partially through the entire matrix, wherein the transfer agent indentations contain transfer agent layers (150) that extend out from the transfer agent indentations; E: Certain embodiments of transfer agent layers (150) that extend out from the surface of a substrate (111); F: Certain embodiments of a matrix (110) and transfer agent layers (150) in transfer agent bulge spaces (153) extending into the matrix at the bulge regions of the matrix.

FIG. 10—FIG. 10 illustrates certain embodiments of the transfer through a sealing layer (160) of a transfer agent layer (150) from a tapered transfer agent indentation (162) extending into a matrix (110), wherein the sealing layer is positioned flush against the face of the matrix opposite the face upon which the pressure source (arrow) is applied during the transfer. FIG. 10A illustrates certain embodiments of a pressure source (arrow) being applied to the transfer agent layer (150) in a tapered transfer agent indentation (162) with a sealing layer (160) on one face of a matrix (111) and a barrier layer (157) on the other face. FIG. 10B illustrates certain embodiments of the transfer of the transfer agent layer (150) through the sealing layer (160). Note that the transfer of the transfer agent layer breaks the sealing layer in the region of the sealing layer aligned with the transfer agent indentation in these embodiments.

FIG. 11—FIG. 11 illustrates certain embodiments of the transfer through a sealing layer of a transfer agent layer from a transfer agent bulge space, wherein the sealing layer is positioned flush against the surface of the face of the matrix opposite the face upon which the pressure source is applied during the transfer. FIG. 11A illustrates certain embodiments of a pressure source (arrow) being applied to a transfer agent layer (150) in a transfer agent bulge space (162) with a sealing layer (160) on one face of a matrix (110). FIG. 11B illustrates certain embodiments of the transfer of the transfer agent layer (150) through the sealing layer (160) resulting from the application of the pressure source as shown in FIG. 11A. Note that the transfer of the transfer agent layer breaks the sealing layer in the region of the sealing layer aligned with the transfer agent bulge space in these embodiments.

FIG. 12—FIG. 12 depicts certain embodiments of genetic material attached to a 96-well plate. FIG. 12A shows certain embodiments of genetic material attached to a 96-well plate (130). FIG. 12B shows certain embodiments of genetic material (e.g. 180) attached as a dried deposit at the bottom of each of three wells (e.g. 134) of a 96-well plate (130). FIG. 12C shows certain embodiments of genetic material in solution (190) sealed into each of three wells (e.g. 134) of a 96-well plate (130) by a sealing layer (170).

### DETAILED DESCRIPTION OF EXEMPLARY EMBODIMENTS

It is to be understood that both the foregoing general description and the following detailed description are exemplary and explanatory only and are not restrictive of the invention, as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, use of the term “portion of x” includes a part of x or all of x.

The section headings used herein are for organizational purposes only and are not to be construed as limiting the subject matter described. All documents and portions of documents cited in this application including but not limited to patents, patent applications, articles, books, and treatises, are hereby expressly incorporated by reference in their entirety for any purpose.

### DEFINITIONS

Alignable—as used herein, refers to the ability to align one object to another.

Attach—as used herein, refers to placing material onto or into a substrate such that the material substantially maintains its position at least until positive measures are taken to remove it from the substrate. Attached—as used herein, refers to material that has been deposited onto or into a substrate such that the material substantially maintains its position at least until positive measures are taken to remove it from the substrate.

The word “attach” includes “chemically attach” in which a material is chemically bonded to a substrate. The word “attached” includes “chemically attached” which describes material that has been chemically bonded to a substrate. In certain embodiments, chemically attached material on a substrate substantially remains in its placed position when contacted with reaction reagents.

The word “attach” includes physically attaching a material to a substrate. The word “attach” includes physically containing a material in a compartment by, for example, sealing the material in the compartment. The word “attached” may describe material that has been physically attached to a substrate. The word “attached” may describe material that has been physically contained in a compartment by, for example, sealing the material in the compartment.

Cover—as used herein, refers to any object that functions to cover another object. In certain embodiments, a cover includes objects that function to cover at least part of a multiwell plate. A cap is a cover that covers one compartment such as, but not limited to, a well or tube.

Defined population—as used herein, refers to any group of individuals. In certain embodiments, a defined population may include only individuals that share at least one common characteristic. In certain embodiments, a defined population may include a random group of individuals that may or may not share at least one common characteristic.

Deposit—as used herein, refers to the placing of material onto or into a substrate. Deposited—as used herein, refers to material that has been placed onto or into a substrate. The word “deposit” includes the term “attach.” The word “deposited” includes the term “attached.” The word “deposit” may also include placing material into a cavity, such as a capillary, of a substrate. The word “deposited” may also define material that has been placed into a cavity, such as a capillary, of a substrate. In certain embodiments, genetic material may be deposited using contact techniques such as, but not limited to, pin contact printing or library transfer tool transferring. In certain embodiments, genetic material may be deposited using techniques such as, but not limited to, pipetting, syringe transferring, or inkjet techniques such as, but not limited to, piezo ejection, bubble inkjetting, or acoustic inksetting. The term deposit or deposited includes the incorporation of genetic material in a transfer agent layer by, for example, the addition of genetic material to a slurry of the transfer agent used to form the transfer agent layer.

Derive—as used herein, refers to either directly or indirectly obtaining from a source. In certain embodiments, genetic material that derives from an individual is DNA obtained directly from an individual. In certain embodiments, genetic material that derives from an individual is DNA obtained from an amplification of an individual's DNA.

Fix—as used herein, refers to a chemical preservation process to reduce or inhibit enzymatic digestion or degradation of nucleic acids.

Genetic material—as used herein, refers to any substance that contains DNA representative of at least a part of an organism's genome.

Genomic DNA—as used herein, refers to any DNA derived from the nucleus or the mitochondria of a cell.

Hybridization—as used herein, refers to a process wherein a first nucleotide sequence that contains sufficient nucleotide homology to a second nucleotide sequence binds to the second nucleotide sequence. In certain embodiments, hybridization occurs as a step of a process in which a primer or probe selectively binds to a predetermined target genetic sequence.

Transfer agent—as used herein, refers to a substance onto or into which genetic material may be deposited, which can transport the genetic material when the transfer agent is moved. In certain embodiments, a transfer agent is water soluble. In certain embodiments, a transfer agent is substantially inert. In certain embodiments, a transfer agent may comprise a mixture of two or more different substances.

In certain embodiments, a transfer agent has properties of a powder. In certain embodiments, the transfer agent may comprise a sugar such as, but not limited to, a monosaccharide, a disaccharide, and/or a trisaccharide. In certain embodiments, the transfer agent may comprise a sugar other than a monosaccharide, a disaccharide, or a trisaccharide. In certain embodiments, the transfer agent may comprise, but is not limited to, glucose, sucrose, fructose, galactose, and/or mannose. In certain embodiments, the transfer agent is a sugar alcohol such as, but not limited to, dulcitol and/or sorbitol. In certain embodiments, the transfer agent may be a triose, cyclodextran, dextran, and/or a four-carbon sugar. In certain embodiments, the transfer agent may be a polyethylene glycol. In certain embodiments, the transfer agent may comprise a mixture of two or more different powders.

Transfer agent layer—as used herein, refers to a layer comprising a transfer agent. In certain embodiments, a substrate may comprise a plurality of separate transfer agent layers. In certain embodiments, the plurality of separate transfer agent layers may be positioned in a single plane. In certain embodiments, a substrate has a single contiguous transfer agent layer on the substrate. In certain embodiments, upon transfer, a transfer agent layer is removed from the substrate in substantially one piece. In certain embodiments, upon transfer, a transfer agent layer is removed from the substrate in more than one piece.

In certain embodiments, a transfer agent layer is formed from a slurry of a transfer agent. In certain embodiments, a transfer agent layer is not formed from a slurry and may be formed by techniques such as, but not limited to, compression of a dry transfer agent into a transfer agent layer. In certain embodiments, genetic material may be incorporated in a transfer agent layer by addition of genetic material to a slurry of a transfer agent. In certain embodiments, genetic material may be deposited onto a transfer agent layer after the transfer agent layer has been formed.

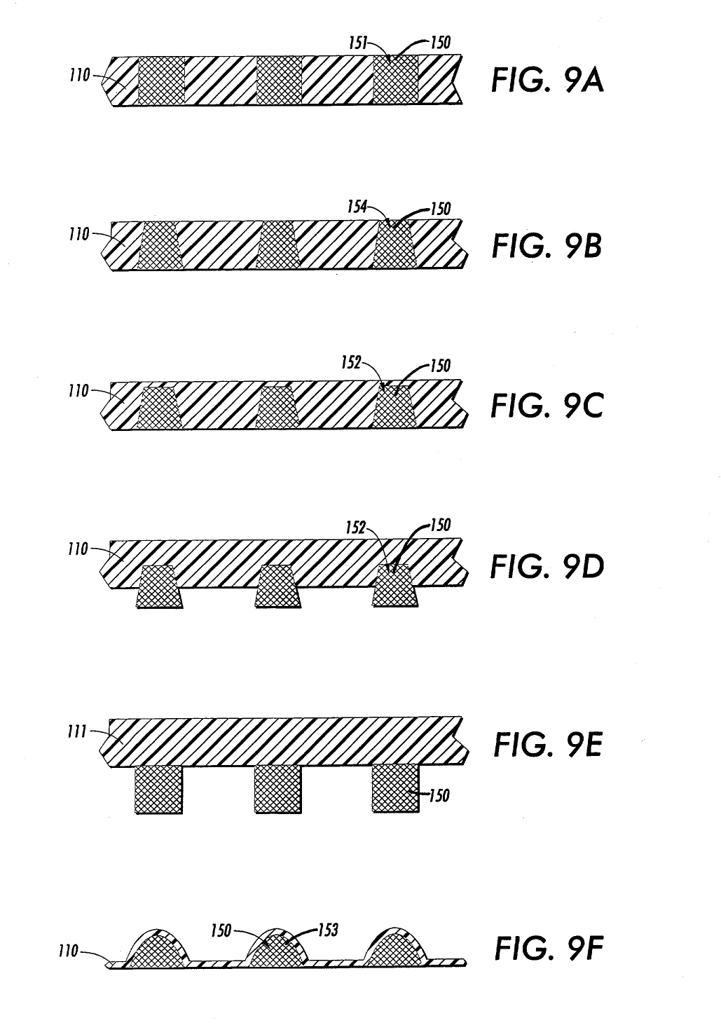

Matrix—as used herein, refers to a material that includes transfer agent spaces. In certain embodiments, a matrix is a material through which, or partially through which, one or more transfer agent spaces may extend. Transfer agent space—as used herein, refers to a region capable of containing at least one transfer agent. In certain embodiments, a transfer agent space may contain a transfer agent. In certain embodiments, a transfer agent space may be completely filled or partially filled. In certain embodiments, a transfer agent space may contain material that extends out from the transfer agent space. In certain embodiments, a transfer agent space may be empty. In certain embodiments, a transfer agent space may contain a substance other than a transfer agent. Transfer agent hole—as used herein, refers to a transfer agent space that extends through the entire matrix. Transfer agent indentation—as used herein, refers to a transfer agent space that only extends partially through the matrix. Transfer agent bulge space—as used herein, refers to a transfer agent space formed by a bulge in the form of a matrix (see, e.g., FIG. 9F).

Pressure source—as used herein, refers to any source that can be used to dislodge a portion of the transfer agent layer such as, but not limited to, a plurality of pins or rods; positive or negative gas pressure such as, but not limited to, air or nitrogen; or liquid pressure such as, but not limited to, water or other aqueous solution or suspension. In certain embodiments, pins or rods used as a pressure source have a width less than or equal to the width of the transfer agent spaces.

Print—as used herein, refers to the process of depositing genetic material onto a substrate using a mechanical mechanism. In certain embodiments, one may use direct contact with a device or indirect application of drops ejected from a reservoir.

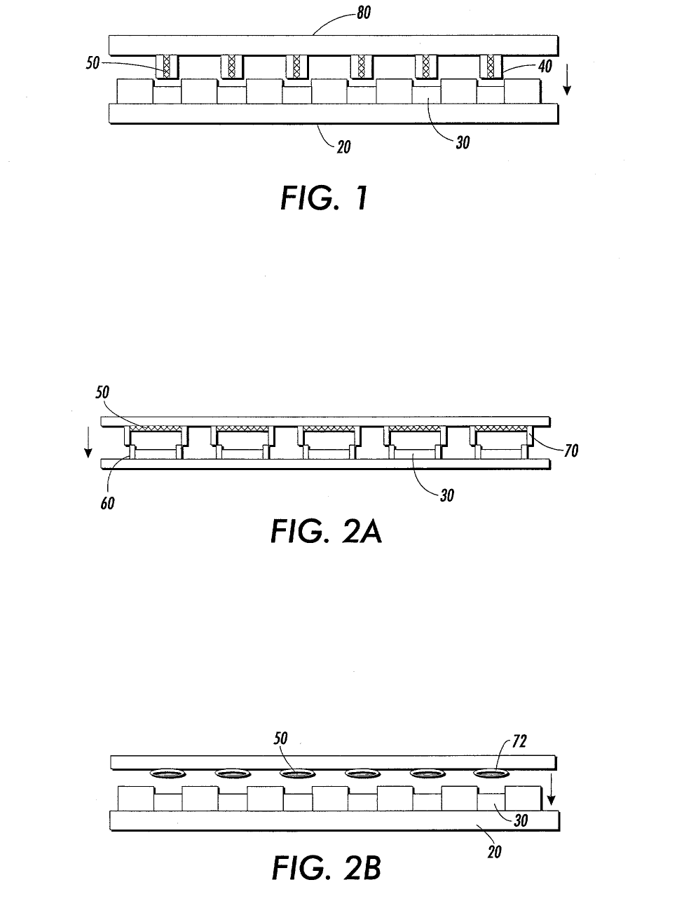

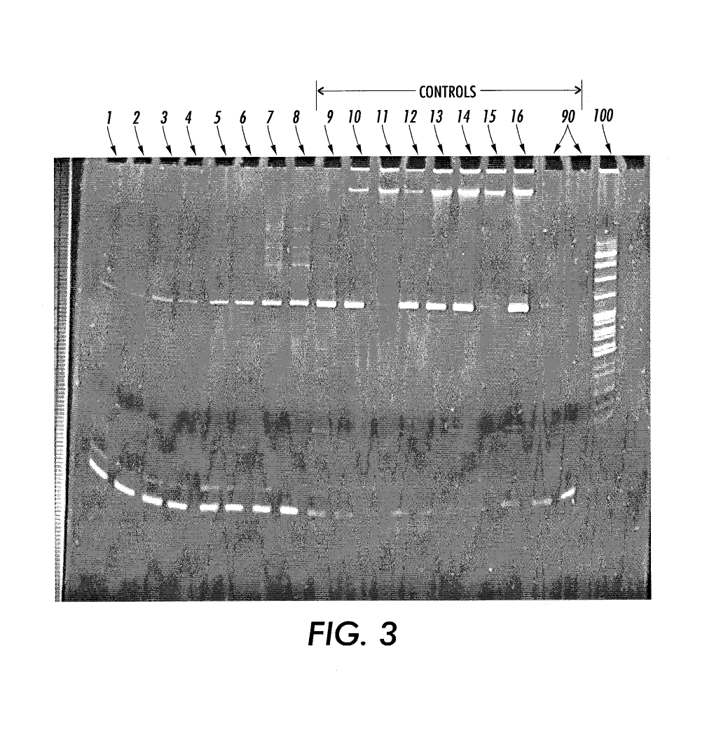

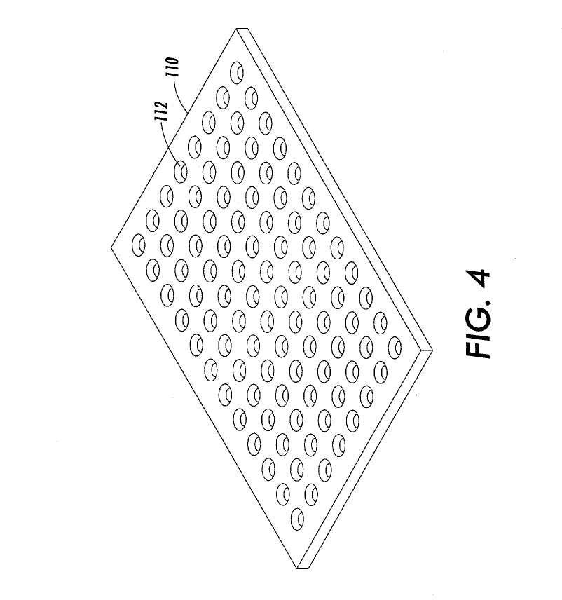

Protrusion—as used herein, refers to an extension of a substrate that extends out from the substrate. In certain embodiments, the protrusion also comprises a capillary within the protrusion that extends at least part of the length of the protrusion. In certain embodiments, the protrusion extends from a cover and the protrusion comprises a capillary within the protrusion that extends at least a part of the length of the protrusion with an opening on the side of the protrusion that faces the item that is to be covered (see, e.g., FIG. 1).

Purified—as used herein, refers to the state of a biological material that has been substantially separated from at least one other material with which it had been intermingled.

Sealing element—as used herein, refers to an object that forms a seal between two objects when in contact with both objects.

Substrate—as used herein, refers to an object onto which genetic material may be deposited. In certain embodiments, the substrate may be, but is not limited to, a multiwell plate, a glass slide, a filter membrane, or a plurality of tubes. In certain embodiments, a substrate comprises at least one transfer agent layer. In certain embodiments, a substrate comprises a matrix and one or more transfer agent layers contained in one or more of the transfer agent spaces. In certain embodiments, the matrix has one or more transfer agent spaces that may extend through a partially through the matrix.

Target compartment—as used herein, refers to a compartment capable of containing transferred genetic material. In certain embodiments, the transferred genetic material is genetic material that has been deposited on or into a transfer agent layer. In certain embodiments, the target compartment is physically separate from other compartments. In certain embodiments, a target compartment includes, but is not limited to, a well of a multiwell plate or a tube.

### EXEMPLARY EMBODIMENTS

The present invention is directed to devices and methods for analyzing genetic material. In certain embodiments, genetic material is deposited on a substrate. In certain embodiments, a substrate comprises a matrix, one or more transfer agent spaces, and one or more transfer agent layers contained in one or more of the transfer agent spaces.

In certain embodiments, at least one device comprises a substrate; and a first genetic material position and a second genetic material position on the substrate that each comprise genetic material attached to the substrate, wherein the genetic material of the first genetic material position comprises purified genomic DNA comprising more than one chromosome of the genomic DNA from at least one cell of an individual within a defined population and the genetic material of the second genetic material position comprises purified genomic DNA comprising more than one chromosome of the genomic DNA from at least one cell of an individual within a defined population, and wherein the genetic material of the first genetic material position is separate from the genetic material of the second genetic material position.

In certain embodiments, methods of distributing genetic material comprise distributing at least one device to at least one recipient. In certain embodiments, the at least one device comprises a substrate comprising a matrix; one or more separate transfer agent spaces that extend through or partially through the matrix; one or more transfer agent layers contained in one or more of the transfer agent spaces. In certain embodiments, these methods comprise distributing at least one device to at least one recipient.

### EXEMPLARY USES OF THE INVENTION

Various embodiments may be used for different purposes. For example, in certain embodiments, one may use the invention to determine the frequency of polymorphisms, alleles, and/or mutations in a population. In certain embodiments, one may use the invention to correlate polymorphisms, alleles, or mutations with a particular condition, e.g., a disease phenotype. In certain embodiments, one may use the invention to determine a certain sequence or sequences of deposited genetic material from individuals within a defined population.

In certain embodiments, a defined population to be studied is defined in view of certain characteristics such as, but not limited to, geographical location, age, sex, family history, disease state, predisposition to disease, and ethnicity. In certain embodiments, the defined population is composed of human beings displaying a common phenotype, e.g., human beings with a particular disease. Such diseases may include, but are not limited to, hypertension, cancer, heart disease, neurological diseases, mental diseases, and infectious diseases. In certain embodiments, the population is composed of mice displaying a common phenotype. In certain embodiments, the population is composed of human beings between any age range, for example, between the ages of 65 years old and 75 years old, or between the ages of 40 and 80 years old, or between the ages of 20 years old and 30 years old.

In certain embodiments, the invention allows one to analyze separate genetic material from a predetermined number of individuals within the defined population.

Exemplary Genetic Material

In certain embodiments, sources of genetic material may include, but are not limited to, vertebrate, invertebrate, plant, and prokaryotic sources. In certain embodiments, sources of genetic material may include, but are not limited to, mammals, such as primates, rodents, and farm animals. In certain embodiments, sources of genetic material may include, but are not limited to, humans and mice.

In certain embodiments, genetic material may be obtained from tissues such as, but not limited to, blood, skin, mucosal tissue, biopsy samples, tumors, warts, hair, and other tissues, and cultured cell lines deriving from individuals in a defined population.

In certain embodiments, genetic material may be purified by methods such as, but not limited to, those described in Sambrook and Russell, Molecular Cloning: A Laboratory Manual, 3^{rd} Edition, Chapter 6, incorporated herein by reference in its entirety for any purpose. In certain embodiments, genetic material may be purified using commercial kits such as, but not limited to, PUREGENE DNA Isolation Kit (Gentra Systems, Minneapolis, Minn.), GenElute Mammalian Genomic DNA Purification Kit (Sigma-Aldrich), and Wizard Genomic DNA Purification Kit (Promega, Madison, Wis.). In certain embodiments, genetic material may be purified using a robotic system such as, but not limited to, the MultiPROBE II HT EX (Perkin Elmer, Boston, Mass.). In certain embodiments, care should be taken to avoid contamination between samples.

In certain embodiments, purified genetic material may comprise at least 25%, or at least 50%, or at least 75%, or at least 80%, or at least 90%, or at least 95% of the genomic DNA from at least one cell of an individual within a defined population. In certain embodiments, purified genetic material may comprise the entire genomic DNA from at least one cell of an individual within a defined population.

In certain embodiments, genetic material may comprise more than one chromosome of the genomic DNA from at least one cell of an individual within a defined population. In certain embodiments, genetic material may comprise more than two chromosomes of the genomic DNA from at least one cell of an individual within a defined population. In certain embodiments, genetic material may comprise more than ten chromosomes of the genomic DNA from at least one cell of an individual within a defined population. In certain embodiments, genetic material may comprise more than twenty chromosomes of the genomic DNA from at least one cell of an individual within a defined population. In certain embodiments, genetic material may comprise more than twenty pairs of chromosomes of the genomic DNA from at least one cell of an individual within a defined population. In certain embodiments, genetic material may comprise all twenty three pairs of chromosomes of the genomic DNA from at least one cell of an individual within a defined population.

In certain embodiments, samples of tissues or cells may be frozen or fixed, which may provide longer storage times. In certain embodiments, genetic material attached to or deposited on a substrate may be frozen or fixed, which may provide longer storage times.

In certain embodiments, genetic material is amplified prior to deposition on a substrate. In certain embodiments, genetic material is amplified by techniques such as, but not limited to, the polymerase chain reaction (PCR); Rolling Cycle Amplification Technology (RCAT) (Lizardi et al., Nature Genetics, 19(3):225-232 (1998)); Ligase Chain Reaction (Wiedmann et al., PCR-METHODS AND APPLICATIONS, V3 N4:S51-S64 (1994)); and Strand Displacement Amplification (Nadeau et al., Analytical Biochemistry 276(2):177-187 (1999)), all of which are hereby expressly incorporated by reference in their entirety for any purpose.

Exemplary Substrates

Substrates that may be used for certain embodiments include, but are not limited to, filter membranes, glass substrates, paper, SU8, poly(dimethyl siloxane), polystyrene, polypropylene, acrylamide, cellulose, glass, polyethylene vinyl acetate, polymethacrylate, polyethylene, polyethylene oxide, polysilicates, polycarbonates, teflon, fluorocarbons, silicon rubber, polyanhydrides, polyglycolic acid, polylactic acid, polyorthoesters, polypropylfumerate, collagen, glycosaminoglycans, polyamino acids, metal, and plastic substrates. According to certain embodiments, filter membranes may be composed of materials that include, but are not limited to, nitrocellulose and nylon.

In certain embodiments, the substrate may be a multiwell plate. In certain embodiments, multiwell plates include, but are not limited to, plates that include more than one separate well in which separate reactions may occur. In certain embodiments, the reactions include, but are not limited to, hybridizations, product amplification reactions, signal amplification reactions, sequencing reactions, and restriction enzyme digestions. In various embodiments, any number of wells may be provided in the multiwell plate. In certain embodiments, the multiwell plates include, but are not limited to, 96-well-, 384-well-, 1536-well-, and 6144 well-plates. No upper limit on the number of wells on a multiwell plate is envisioned. In certain embodiments, the wells of the multiwell plate may be microwells. In various embodiments, multiwell plates may be made of glass, plastic, metal, or other materials.

In certain embodiments, high density microwell plates may be used. In certain embodiments, microwell plates may comprise several thousand, several ten's of thousands, or several 100's of thousands of wells. In certain embodiments, such microwell plates may be made with a polymer, including, but not limited, to SU-8, polystyrene, methylacrylate, polypropylene and poly(dimethyl siloxane). In certain embodiments, microwell plates may be made with a metal, including, but not limited to, stainless steel, aluminum and nickel.

In certain embodiments, one may employ techniques including, but not limited to, microlithography, plastic molding, stamping, hot-pressing, laser machining, and conventional machining to make the multiwell plates from polymers. In certain embodiments, one may employ techniques including, but not limited to, microlithography, stamping, Electro Discharge Machining, laser machining, conventional metal machining to make multiwell plates from metals. In certain embodiments, the wells may be separated by 100 to 200 microns.

In certain embodiments, a substrate is coated with an agent that provides a positive charge such as, but not limited to, poly-L-lysine or amino silane.

In certain embodiments, a substrate may comprise a cover for a multiwell plate. In certain embodiments, the cover may comprise an area sufficient to cover an entire multiwell plate. In certain embodiments, the cover may comprise an area sufficient to cover at least a majority of a multiwell plate. In certain embodiments, the cover may comprise an area sufficient to cover at least two wells of a multiwell plate.

In certain embodiments, genetic material may be deposited at positions on the cover such that there are at least two positions, and that each position corresponds to a different well of a multiwell plate. In certain embodiments, genetic material may be deposited at positions on the cover such that there are at least ten positions, and that each position corresponds to a different well of a multiwell plate. In certain embodiments, genetic material may be deposited at positions on the cover such that there are at least 96 positions, and that each position corresponds to a different well of a multiwell plate. In certain embodiments, genetic material may be deposited at positions on the cover such that there are at least 384 positions, and that each position corresponds to a different well of a multiwell plate.

In certain embodiments, the substrate may comprise at least two sealing elements, each of which may be placed around a different deposited position of genetic material on the cover. In certain embodiments, when such a cover is used as a lid for a multiwell plate, a seal may form between the cover and the wells of the multiwell plate that are positioned opposite the deposited positions of genetic material on the cover (see, e.g., FIG. 2B). In certain embodiments, the substrate may comprise a sufficient number of sealing elements such that a majority of the wells of a multiwell plate will be sealed. In certain embodiments, the substrate comprises a sufficient number of sealing elements such that substantially all of the wells of a multiwell plate will be sealed.

In certain embodiments, tape may be used to hold a cover and a multiwell plate together. In certain embodiments, tape may be used to hold a cover with no sealing elements and a multiwell plate together such that the wells of the plate are sealed by the cover.

In certain embodiments, the cover for the multiwell plate may be constructed of material that permits the transmission of light, such as UV light, sufficient to allow the transmission of sufficient excitation light to produce detectable emission light. This material includes, but is not limited to, quartz and poly (dimethylsiloxane). In certain embodiments, light of wavelengths of at least 250 nm or higher may be transmitted through the material.

In certain embodiments, the substrate material does not substantially interfere with the detection of radioactivity.

In certain embodiments, optically transparent areas will not constitute the entire cover, but may be located above at least a majority of the wells where genetic material will be analyzed. In certain embodiments, the optically transparent areas may be located above at least two wells of a multiwell plate. In certain embodiments, the optically transparent areas are large enough to allow the transmission and collection of sufficient light signal for detection of the light signal.

In certain embodiments, at least a portion of the wells, such as the bottom of the wells, of a multiwell plate may be constructed of a material that permits transmission of light, such as UV light, sufficient to allow the transmission of sufficient excitation light to produce detectable emission light. This material includes, but is not limited to, quartz and poly (dimethylsiloxane). In certain embodiments, light with a wavelength of at least 250 nm may be transmitted through the material. In certain embodiments, the optically transparent areas will not constitute the entire bottom of the wells, but will only be located at locations where the genetic material will be deposited. In certain embodiments, the optically transparent areas are large enough to allow the transmission and collection of sufficient light for detection of a light signal. In certain embodiments, the multiwell plate may be a commercial product such as, but not limited to, UVStar or μClear Plates (Greiner Bio-One, Germany).

In certain embodiments, a substrate may comprise a cover for a multiwell plate configured such that the cover contains at least two protrusions, each of which corresponds with a well of a multiwell plate (see e.g. FIG. 1). In certain embodiments, at least two of the protrusions extend to a depth sufficient to place at least a portion of the protrusion in contact with a predetermined volume in the wells.

In certain embodiments, the protrusions may contain a capillary or a slit into which genetic material may be deposited. In certain embodiments, the capillary or slit has a cylindrical shape. In certain embodiments, the capillary or slit has a non-cylindrical shape. In certain embodiments, the capillary or slit extends the length of a protrusion. In certain embodiments, the capillary or slit extends at least a majority of the length of a protrusion. In certain embodiments, the capillary or slit extends less than a majority of the length of a protrusion.

In certain embodiments, a cover with at least two protrusions is manufactured by use of a photo-imageable polymer such as, but not limited to, SU-8. In certain embodiments, a cover with at least two protrusions may be manufactured using a precision mold master such as, but not limited to, a plastic optical lens. In certain embodiments, a cover with at least two protrusions may be manufactured using injection molded parts.

In certain of these embodiments, a sealing element may be located around the protrusions in a configuration such that when the cover is placed on multiwell plate, a seal will form between the cover and the wells of the multiwell plate. In certain of these embodiments, one may manipulate the cover and the multiwell plate such that at least a portion of the genetic material becomes detached from the cover so that it is included in a reaction volume in the wells. In certain such embodiments, the manipulation may be shaking or centrifugation. In certain embodiments, genetic material may become detached by the inherent reflux of the PCR liquid during the high temperature portions of a PCR cycle.

In certain embodiments, the substrate comprises at least two caps that fit at least two wells of a multiwell plate. In certain embodiments, the substrate comprises a sufficient number of caps to at least correspond to one row or column of wells of a multiwell plate. In certain embodiments, the substrate comprises a sufficient number of caps to correspond to at least substantially all wells of a multiwell plate. In certain embodiments, these caps are provided in a strip format such that they cover at least one row of wells. In certain embodiments, these caps are in a two dimensional format such that they cover more than two rows of wells.

In certain embodiments, the substrate is a cover that comprises one or more holes corresponding to each position of deposited genetic material. The holes extend through the cover from the top of the cover to the bottom of the cover where the genetic material is deposited, such that the deposited material covers the holes. In certain embodiments, a positive pressure source may be applied to the holes at the top of the cover to cause transfer of the genetic material from the cover.

In certain embodiments, the pressure source includes a gas pressure source and/or a liquid pressure source.

Gasses that may be used for the gas pressure source include, but are not limited to, air and nitrogen. In certain embodiments, the gas used for the gas pressure source may comprise a mixture of two or more different gases.

Liquids that may be used for the liquid pressure source include, but are not limited to, water and other aqueous solutions or suspensions. In certain embodiments, the liquid used for the liquid pressure source may comprise a mixture of two or more different liquids. In certain embodiments, liquid used for the liquid pressure source comprises reaction reagents such as, but not limited to, PCR reagents. In certain embodiments, liquid used for the liquid pressure source do not comprise reaction reagents.

In certain embodiments, there may be a layer between the substrate and the deposited genetic material. In certain embodiments, the layer may be material that helps to release the deposited genetic material from the substrate when one wants to proceed with a reaction. In certain embodiments, the layer dissolves when contacted by certain reagents, releasing the deposited genetic material from the substrate.

In certain embodiments, a substrate may comprise one or more transfer agent layers. In certain embodiments, one or more of the transfer agent layers are each attached to the substrate. FIG. 9E shows certain embodiments in which transfer agent layers are attached to the substrate.

In certain embodiments, a substrate is a matrix that includes one or more transfer agent spaces. In certain embodiments, a transfer agent space is shaped as a transfer agent hole where the transfer agent space extends through the entire matrix (see, e.g., FIG. 9A). In certain embodiments, a transfer agent space is shaped as an indentation where the transfer agent space only extends partially through the matrix (see, e.g., FIG. 9C). In certain embodiments, a transfer space indentation is configured such that a portion of the matrix disposed above the transfer agent indentation (see, e.g., FIG. 9C) is sufficiently deformable such that a pressure source can be applied to dislodge a portion of the transfer agent layer from the indentation.

In certain embodiments, the edges of a transfer agent space may be tapered such that the width of the transfer agent space on one face of the matrix is larger that the width of the transfer agent space on the other face of the matrix (see, e.g., FIG. 9B). In certain embodiments, the larger width side of a transfer agent space may be the side that faces the target compartment during transfer of the transfer agent layer to the target compartment (see, e.g., FIG. 9B).

In certain embodiments, a transfer agent space may be circular in shape. In certain embodiments, a transfer agent space may be non-circular in shape. In certain embodiments, a transfer agent space may be rectangular in shape. In certain embodiments, a transfer agent space may be irregularly shaped.

In certain embodiments, a matrix includes one or more transfer agent spaces that do not contain transfer agent layers. In certain embodiments, at least a majority of the transfer agent spaces contain genetic material.

In certain embodiments, a substrate comprising a matrix with one or more transfer agent spaces includes one or more transfer agent layers. In certain embodiments, at least a majority of the transfer agent layers are each attached to the matrix at a transfer agent space.

In certain embodiments, at least a majority of the transfer agent layers extend out from the transfer agent spaces that contain the transfer agent layers ( see e.g., FIG. 9D). In certain embodiments, all of the transfer agent layers extend out from the transfer agent spaces that contain the transfer agent layers.

In certain embodiments, a matrix may be configured as a flat or substantially flat layer. In certain embodiments, a matrix may be configured as a flat or substantially flat layer with one or more bulge regions in which the matrix bulges out from the plane of the flat or substantially flat portion of the matrix (see, e.g., 9F). In certain embodiments, a matrix may be configured as a non-flat layer with one or more bulge regions in which the matrix bulges out in two or more positions on the layer. In these embodiments, the bulging out of the matrix in the bulge region creates a transfer agent bulge space. In certain embodiments, the transfer agent bulge space contains a transfer agent layer.

In certain embodiments, a matrix may be made from a variety of materials such as, but not limited to, filter membranes, glass substrates, paper, SU8, poly(dimethyl siloxane), polystyrene, polypropylene, acrylamide, cellulose, nitrocellulose, glass, polyethylene vinyl acetate, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, polyamino acids and plastic substrates. In certain embodiments, a matrix may be made of a metal such as, but not limited to, aluminum, copper, and stainless steel. In certain embodiments, a matrix may be made from materials such as wax or other low-temperature melting materials.

In certain embodiments, a matrix may be between 0.1 and 100 millimeters thick. In certain embodiments, a matrix may be a metal and may be between 0.1 and 10 millimeters thick. In certain embodiments, a metal matrix may be less than 0.1 or more than 10 millimeters thick.

In certain embodiments, a matrix may be plastic and may be between 1 and 100 millimeters thick. In certain embodiments, a plastic matrix may be less than 1 or more than 100 millimeters thick.

In certain embodiments, a matrix may have about the same width and length dimensions as a 96- or 384-well plate. In certain embodiments, a matrix may have different width and length dimensions than a 96- or 384-well plate. In certain embodiments, a matrix may have a substantially greater length than width dimension and, therefore, be shaped as a strip. In certain embodiments, a matrix may be substantially circular in shape.

In certain embodiments, the positions of at least two of the transfer agent spaces within a matrix are arranged such that each transfer agent space is alignable with a separate single target compartment. In certain embodiments, the positions of a majority of the transfer agent spaces within the matrix are arranged such that each transfer agent space is alignable with a separate single target compartment. In certain embodiments, the positions of all of the transfer agent spaces within the matrix are arranged such that each transfer agent space is alignable with a separate single target compartment.

In certain embodiments, the positions of at least two of the transfer agent spaces are arranged such that each transfer agent space is alignable with a separate single well of a 96-, 384-, or 1536-well plate. In certain embodiments, the positions of at least two of the transfer agent spaces are arranged such that each transfer agent space is alignable with a separate single well of a multiwell plate other than a 96-, 384-, or 1536-well plate.

In certain embodiments, each of at least two transfer agent spaces has a smaller width than the width of a target compartment to which it is alignable. In certain embodiments, such an arrangement may reduce contamination of non-aligned target compartments when transfer agent layers are transferred from the matrix to their aligned target compartments.

In certain embodiments, 96 or 384 transfer agent spaces extend into a matrix. In certain embodiments, each of the 96 or 384 transfer agent spaces is alignable with a separate well of a 96- or 384-well plate, respectively. In certain of these embodiments, the width of the transfer agent spaces are less than or equal to the width of their aligned wells of the plate. In certain of these embodiments, the transfer agent spaces are circular in shape.

In certain embodiments, a matrix comprises at least one transfer agent space that contains transfer agent. In certain embodiments, a plurality of separate transfer agent spaces contain transfer agent. In certain embodiments, the separate transfer agent spaces may be positioned in a single plane in the matrix.

In certain embodiments, a transfer agent layer may be contained in a transfer agent space of a matrix. In certain embodiments, a plurality of transfer agent layers may be contained in a plurality of separate transfer agent spaces. In certain embodiments, the separate transfer agent layers may be positioned in a single plane in the matrix. In certain embodiments, a substrate comprises a single contiguous transfer agent layer on the surface of the substrate.

In certain embodiments, a transfer agent layer may be between 0.1 to 100 millimeters in thickness. In certain embodiments, a transfer agent layer may be less thick than the matrix. In certain embodiments, a transfer agent layer may be the same thickness as, or of greater thickness than, the matrix.

In certain embodiments, a transfer agent layer comprises a transfer agent such as, but not limited to, a sugar. In certain embodiments, the transfer agent is water soluble. In certain embodiments, the transfer agent is substantially inert. In certain embodiments, the transfer agent comprises a substance other than a sugar. In certain embodiments, the transfer agent may comprise, but is not limited to, a low-density polysaccharide such as a monosaccharide, disaccharide, and/or trisaccharide. In certain embodiments, the transfer agent may be sucrose, glucose, fructose, galactose, and/or mannose. In certain embodiments, the transfer agent is a sugar alcohol such as, but not limited to, dulcitol and/or sorbitol. In certain embodiments, the transfer agent may be a triose, cyclodextran, dextran, and/or a four-carbon sugar. In certain embodiments, the transfer agent may be a polyethylene glycol.

In certain embodiments, a transfer agent comprises a mixture of two or more different substances. Such multiple substances may include, but are not limited to, glucose and sucrose.

In certain embodiments, a transfer agent may be formed into a transfer agent layer in a transfer agent space by mixing the transfer agent with a liquid such as, but not limited to, water or a buffered aqueous solution to obtain a slurry. The slurry may then be spread into a transfer agent space and then dried, or allowed to dry, such that the transfer agent solidifies into a layer. In certain embodiments, a flat-edged instrument may be used to scrap away excess transfer agent from the transfer agent space.

In certain embodiments, a slurry of a transfer agent may have a density between, but not limited to, 1 and 10 grams per milliliter. In certain embodiments, the slurry may have a density between 6 and 7 grams per milliliter.

In certain embodiments, transfer agent layers are formed as follows. A matrix with transfer agent holes extending through the matrix is placed flat upon a Teflon-coated surface. A slurry of sucrose in water at about 6.6 grams/ml is then spread over the transfer agent spaces. The slurry is allowed to dry. A flat-edged instrument, such as a knife, is then used to scrape horizontally across the matrix such that excess slurry is removed from the matrix. After drying, the slurry forms transfer agent layers that are then ready for depositing of genetic material.

In certain embodiments, transfer agent layers are formed in a continuous-feed, mass production manner as follows. A nip of rollers feed substrates and simultaneously press a transfer agent slurry into transfer agent spaces that extend into the matrix. A set of knife-edges, which are positioned downstream from the rollers, scrape away excess slurry.

In certain embodiments, transfer agent layers are formed by pressing dry transfer agent into a transfer agent layer without use of a slurry. In certain embodiments, the transfer agent is pressed into the transfer agent layers with a tool such as, but not limited to, a set of pins. In certain of these embodiments, excess transfer agent is subsequently removed from the substrate by techniques including, but not limited to, blowing, shaking, wiping, and gravity.

In certain embodiments, a transfer agent layer may be formed by directly depositing transfer agent slurry into a transfer agent space that extends into a matrix. In certain of these embodiments, the transfer agent slurry may be dispensed using a syringe.

Exemplary Methods of Depositing Genetic Material on the Substrate

In certain embodiments, genetic material may be deposited onto the substrate using techniques such as, but not limited to, pin contact printing (See, e.g., Schena et al. (1995), Science, 270(5235):467-70; Eisen et al. (1999), Methods Enzymol 303:179-205, both hereby expressly incorporated by reference in their entirety for any purpose). In certain embodiments, genetic material may be deposited using a robotic system using pin contact printing. In certain embodiments, the robotic pin contact printing system may include, but is not limited to, SpotBot Personal Microarrayer (Telechem International, Inc., Sunnyvale, Calif.) or OmniGrid (GeneMachines, San Carlos, Calif.). In certain embodiments, genetic material may be deposited using techniques such as pipetting or syringe transfer.

In certain embodiments, genetic material may be deposited using library transfer tool techniques. In certain embodiments, library transfer tool techniques may employ a device that can deposit several separate volumes of material to distinct different positions on a substrate. In certain embodiments, the device comprises separate pins that each can deposit separate volumes of material onto a substrate. Commercial library transfer tools include, but are not limited to, Slot Pin Replicators (V & P Scientific, Inc. San Diego, Calif.). In certain embodiments, genetic material may be transferred using library transfer tool techniques to a multiwell plate such as, but not limited to, a 96- or 364-well plate.

In certain embodiments, genetic material may be deposited onto the substrate using techniques such as inkjet printing methods (See, e.g., U.S. Pat. No. 6,079,283; “Ink-Jet-Deposited Microspot Arrays of DNA and other Bioactive Molecules,” Methods in Molecular Biology, vol 170: DNA Arrays: Methods and Protocols, edited by J. B. Rampal (Humana Press Inc.), both hereby expressly incorporated by reference in their entirety for any purpose) and other transfer technology that involves the ejection of material onto a substrate.

In certain embodiments, genetic material may be deposited with a commercial system such as SpotArray Enterprise (PerkinElmer, Boston, Mass.) or synQUAD (Cartesian Technologies, Irvine, Calif.). In certain embodiments, genetic material may be deposited by biofluid drop ejection using a multi-ejector system. An exemplary discussion of this technology is described in U.S. patent application Ser. Nos. 09/724,987, filed Nov. 22, 2000, and 09/721,389, filed Nov. 22, 2000, all of which are hereby expressly incorporated by reference in their entirety for any purpose). In certain embodiments, biofluid drop ejection methods provide for the deposit of genetic material contained in small volumes of liquid.

In certain embodiments, genetic material may be incorporated into a transfer agent layer by the addition of genetic material to a slurry of the transfer agent, which may then be formed into a transfer agent layer.

In certain embodiments, the concentration of the genetic material to be deposited (printing solution) may be between 100 nanograms per milliliter to 1 milligram per milliliter. In certain embodiments, the concentration of the printing solution may be between 1 microgram per milliliter and 500 micrograms per milliliter. In certain embodiments, the concentration of the printing solution may be about 50 micrograms per milliliter. In certain embodiments, genetic material is dissolved in aqueous solvents such as, but not limited to, water or buffer such as TE (10 mM Tris.Cl, pH=7.4, 1 mM EDTA). In certain embodiments, dissolution of the genetic material in water may minimize the amount of additives that potentially could interfere with subsequent reactions.

In certain embodiments, genetic material may be deposited onto the substrate in multiple drops per position. In certain embodiments, more than 10 drops of solution containing genetic material are deposited per position. In certain embodiments, more than 100 drops of solution containing genetic material are deposited per position. In certain embodiments, more than 1000 drops of solution containing genetic material are deposited per position.

In certain embodiments, the concentration of genetic material can be normalized between different samples such that the amount of deposited genetic material on the substrate for each sample is approximately equal.

In certain embodiments, genetic material is denatured before, during, or after being deposited on a substrate. In certain embodiments, one denatures the deposited genetic material by submerging into boiling water for 5 minutes. In certain of these embodiments, deposited genetic material is then dried after the boiling.

In certain embodiments, genetic material is deposited on a cover for a multiwell plate. In certain embodiments, genetic material may be deposited on protrusions emanating from a flat substrate that can act as a cover for a multiwell plate, wherein the protrusions correspond to each well of the multiwell plate (see, e.g., FIG. 1). In certain embodiments, genetic material may be deposited on protrusions emanating from a flat substrate that can act as a cover for a multiwell plate, wherein the protrusions correspond to substantially all of the wells of the multiwell plate. In certain embodiments, genetic material may be deposited on protrusions emanating from a flat substrate that can act as a cover for a multiwell plate, wherein the protrusions correspond to at least the majority of the wells of the multiwell plate. In certain embodiments, genetic material may be deposited on protrusions emanating from a flat substrate that can act as a cover for a multiwell plate, wherein the protrusions correspond to at least two wells of the multiwell plate. For certain of these embodiments, the attachment process should be accurate such that each protrusion includes genetic material from only one individual sample.

In certain embodiments, genetic material is deposited into the capillaries of at least two protrusions of a cover with multiple protrusions. In certain embodiments, a solution of genetic material is transferred to at least two protrusions by methods such as, but not limited to, library transfer tool, pin contact printing, or inkjet printing methods. In certain embodiments, the solution containing genetic material is drawn into the capillary by capillary action when the solution comes into contact with the open end of the capillary. In certain embodiments, genetic material stays in solution while in the capillary. In certain embodiments, genetic material is dried in the capillary.

In certain embodiments, the open end of the capillaries of at least two protrusions may be sealed to inhibit or to substantially prevent evaporation, leakage, and/or contamination. In certain embodiments, a standard well-plate sealer, such as, but not limited to, a CycleSeal Plate Sealer (Robbins Scientifics) or a CycleFoil Plate Sealer and Roller (Robbins Scientifics), is used to seal the capillaries. In certain embodiments, sealing is performed by taping the plate lid with a sealing layer and applying pressure.

In certain embodiments, genetic material may be deposited on caps that can be fitted onto wells of a multiwell plate. In certain embodiments, the caps may be connected in a strip format.

In certain embodiments, genetic material may be deposited on at least two wells of a multiwell plate. In certain embodiments, genetic material may be deposited on a majority of the wells of a multiwell plate. In certain embodiments, genetic material may be deposited on substantially all wells of a multiwell plate. In certain embodiments, genetic material may be deposited on all wells of a multiwell plate.

In certain embodiments, genetic material may be deposited on wells of a multiwell plate as a solution. In certain embodiments, deposited genetic material solution may be sealed into the wells by a sealing layer attached to the multiwell plate after deposition. In certain embodiments, two or more multiwell plates, each containing two or more wells with genetic material solution sealed into them may be sold and/or distributed to two or more users. In certain embodiments, two or more multiwell plates, each with a majority of their wells containing genetic material solution sealed into them, may be sold and/or distributed to two or more users. In certain embodiments, two or more multiwell plates, each with substantially all of their wells containing genetic material solution sealed into them, may be sold and/or distributed to two or more users.

In certain embodiments, PCR reagents may be deposited on the wells of a multiwell plate with the deposited genetic material. In certain embodiments, the multiwell plate includes deposited PCR reagents and no genetic material and is used as a control. In certain embodiments, the wells may be sealed after the reaction reagents and genetic material have been deposited and dried. In certain embodiments, the wells are not sealed after reaction reagents and genetic material have been deposited and dried.

In certain embodiments, the deposited genetic material is attached to the substrate. In certain embodiments, attachment of genetic material to a substrate facilitates the storage and/or distribution of that genetic material. In certain embodiments, the deposited genetic material is not attached to the substrate.

In certain embodiments, genetic material may be attached by drying after being deposited on the substrate. In certain embodiments, substrate with deposited genetic material may be air-dried overnight. In certain embodiments, substrate with deposited genetic material may be placed on a slide dryer (a heated platform). In certain embodiments, the substrate is heated on a slide oven at 55° C.-60° C. for 2 to 3 hours. In certain embodiments, the substrate with deposited genetic material may be vacuum dried in an enclosed chamber or vacuum oven. In certain embodiments, the drying occurs at about 55° C.-60° C.

In certain embodiments, genetic material may be chemically attached onto the substrate by techniques such as, but not limited to, UV cross-linking and heat. In certain embodiments, chemically attaching provides a substrate in which most, if not all, of the material remains attached to the substrate when exposed to solutions such as reaction reagents. In certain embodiments, genetic material is chemically attached to the substrate such that the genetic material will be retained on the substrate during any wash steps in a detection reaction.

In certain of these embodiments, the substrate is treated with a special coating prior to deposit of the genetic material that promotes attachment. In certain embodiments, the substrate is coated with poly-L-lysine or amino silane to facilitate genetic material attachment. In certain of these embodiments, genetic material deposited onto the multiwell plate is air-dried and UV cross-linked to produce covalent binding between the plate and the genetic material. In certain embodiments, blocking of fluorescent background from plate coating can be done by shaking in 0.2% SDS solution for 20 minutes.

Exemplary Multiple Substrates

In certain embodiments, a set of multiple substrates comprising deposited genetic material derived from different individuals within a defined population can be prepared. In certain embodiments, each individual substrate of the set of multiple substrates may contain essentially the same genetic material as the other individual substrates of the set. In certain embodiments, genetic material may be arranged in essentially the same positions on each individual substrate of a set of multiple substrates.

In certain embodiments, multiple substrates with deposited genetic material, each containing essentially the same arrangement of genetic material, may be distributed to one or more recipients. Thus, in certain embodiments, a user or users can analyze genetic material from the same set of individuals in multiple runs using different individual substrates. In certain embodiments, multiple recipients can analyze genetic material derived from the same set of sources. In certain embodiments, a user or users can analyze the same nucleotide sequence in the genetic material deposited on multiple substrates. In certain embodiments, a user or users can analyze different nucleotide sequences in genetic material deposited on multiple substrates.

In certain embodiments, multiple multiwell plates containing genetic material in two or more wells of each of at least a majority of the multiwell plates may be distributed. In certain embodiments, there may be sufficient genetic material in each of a majority of the wells of the multiple multiwell plates to supply genetic material to one or more compartments separate from the original multiwell plates. In certain embodiments, the genetic material contained in wells of multiple distributed multiwell plates is transferred to target compartments separate from the original multiwell plates for subsequent reactions.

Exemplary Transfer of Genetic Material from Substrate

In certain embodiments, genetic material deposited on a substrate may be transferred to a target compartment for a subsequent reaction such as, but not limited to, a detection reaction. In certain embodiments, there are various ways genetic material may be transferred from the substrate.

In certain embodiments, a portion of the genetic material at one position on a substrate may be transferred to a target compartment in which a subsequent reaction may take place. In certain embodiments, substantially all of the genetic material at one position on a substrate may be transferred to a target compartment in which a subsequent reaction may take place. In certain embodiments, a majority of the genetic material at one position on a substrate may be transferred to a target compartment in which a subsequent reaction may take place. In certain embodiments, a minority of the genetic material at one position on a substrate may be transferred to a target compartment in which a subsequent reaction may take place.

In certain embodiments, genetic material that had been dried onto a well of a multiwell plate is subsequently transferred into a liquid subsequent reaction phase by immersion into the liquid phase. In certain embodiments, the dried genetic material on the well is transferred into the liquid phase by immersion into the liquid phase and agitation. In certain embodiments, genetic material in solution or suspension in a multiwell plate well is transferred to one or more target compartments separate from the original multiwell plate by methods including, but not limited to, pipetting and syringe transfer.

In certain embodiments, genetic material may be transferred from a substrate by physically transferring a portion of the substrate that contains the genetic material into a target compartment. In certain embodiments, a portion of a substrate containing genetic material may be punched-out from the rest of the substrate by a pressure source. In certain embodiments, a portion of a paper substrate that has genetic material deposited on it may be punched out into a target compartment such as, but not limited to, a tube or a well of a multiwell plate.

In certain embodiments, a transfer agent layer may be transferred into a target compartment in which subsequent reactions or processing may occur. Target compartments may include, but are not limited to, multiwell plates or tubes.

In certain embodiments, a transfer agent layer may be transferred from a matrix by the application of a pressure source. In certain embodiments, the pressure source is applied to the side of the transfer agent layer opposite an aligned target compartment such that the pressure dislodges at least a portion of the transfer agent layer from the matrix.

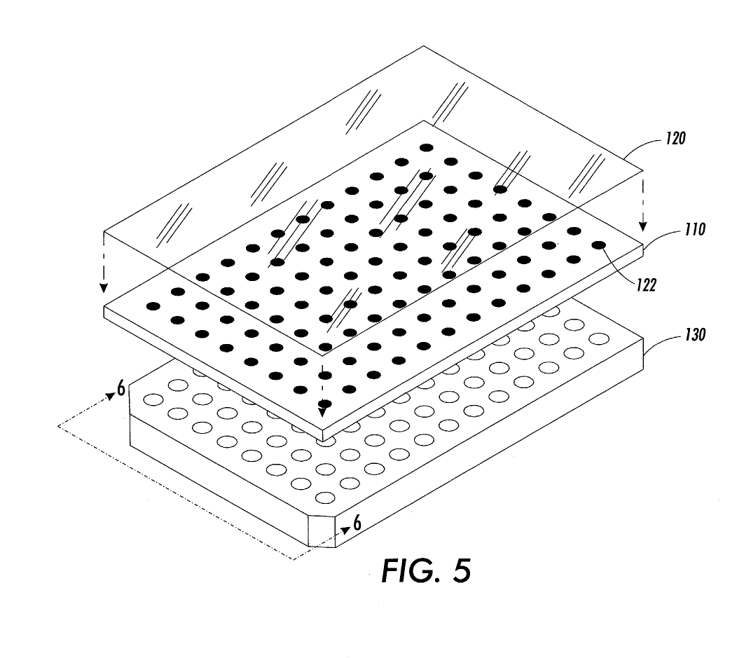

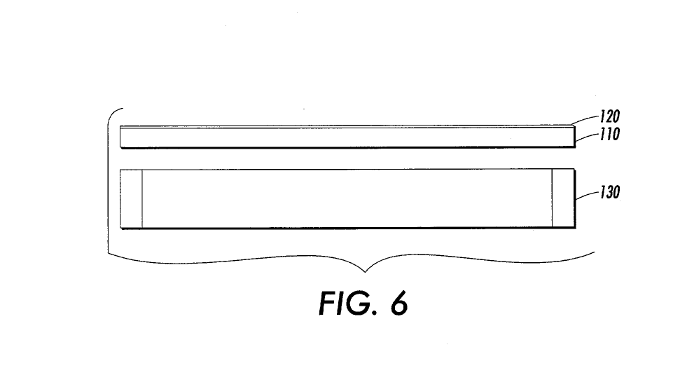

In certain embodiments, in which transfer agent spaces are transfer agent holes that extend all the way through the matrix, a barrier layer may be placed on one side of a matrix, e.g., as shown in FIG. 5. In certain embodiments, the barrier layer substantially inhibits material from the transfer agent layer from contaminating the pressure source used to dislodge a portion of the transfer agent layer from the matrix. In certain embodiments, the barrier layer may comprise, but is not limited to, Mylar, Kapton, or plastic wrap. In certain embodiments, transfer agent indentations extend partially into the matrix. In certain of these embodiments, the portion of the matrix disposed above the indentation may function in the same manner as a barrier layer.

In certain embodiments, a barrier layer may be between 0.1 and 100 millimeters thick. In certain embodiments, a barrier layer may be between 1 and 10 millimeters thick.

In certain embodiments, a pressure source may be a device such as, but not limited to, a rod or a pin. In certain embodiments, a pressure source device contacts a barrier layer or a matrix. In certain embodiments, a pressure source device contacts the transfer agent layer. In certain embodiments, a pressure source device may have a blunt end that contacts the barrier layer, matrix, or the transfer agent layer. In certain embodiments, the blunt ends have slightly rounded edges. In certain embodiments, a pressure source device comprises 96 blunt pins configured such that each pin is alignable with a well of a 96-well plate.

In certain embodiments, a pressure source may be a gas pressure source and/or a liquid pressure source. Gasses that may be used for the gas pressure source include, but are not limited to, air and nitrogen. In certain embodiments, the gas used for the gas pressure source may comprise a mixture of two or more different gases.

Liquids that may be used for the liquid pressure source include, but are not limited to, water and other aqueous solutions or suspensions. In certain embodiments, the liquid used for the liquid pressure source may comprise a mixture of two or more different liquids. In certain embodiments, liquid used for the liquid pressure source comprises reaction reagents such as, but not limited to, PCR reagents. In certain embodiments, liquid used for the liquid pressure source does not comprise reaction reagents. In certain embodiments, no barrier layer is used when a gas and/or liquid pressure source is used to dislodge at least a portion of a transfer agent layer.

In certain embodiments, a pressure source may have a width smaller than the width of the transfer agent spaces. In certain embodiments, a pressure source may have a width the same as or greater than the width of a transfer agent space.

In certain embodiments, the application of a pressure source upon a transfer agent layer dislodges the transfer agent layer as a substantially single piece. In certain embodiments, the application of a pressure source upon a transfer agent layer dislodges a portion of the transfer agent layer. In certain embodiments, the application of a pressure source upon a transfer agent layer causes the dislodged transfer agent layer to fragment into two or more portions. In certain embodiments, the application of a pressure source upon a transfer agent layer causes a portion of the dislodged transfer agent layer to fragment into a powder.

In certain embodiments, genetic material may be transferred from a substrate by buckling out a portion of a matrix that contains the genetic material. In certain of these embodiments, genetic material is contained in a transfer agent bulge space and may be transferred by buckling out the bulge portion of a matrix that contains the genetic material (see, e.g., FIG. 9B). In certain embodiments, a pressure source such as a finger or a roller may be used to buckle out a bulge portion of a matrix that contains a transfer agent layer. In certain of these embodiments, genetic material is contained in one or more transfer agent layers.

In certain embodiments, a contamination shield layer may be placed between the substrate and a plurality of target compartments. The contamination shield layer may reduce contamination of non-aligned target compartments when transfer agent layers are transferred from the substrate to their aligned target compartments. In certain embodiments, a contamination shield layer may be attached to the substrate. In certain embodiments, a contamination shield may not be attached to the substrate.

In certain embodiments, a contamination shield layer may be configured such that it comprises holes. In certain embodiments, at least two of the holes are positioned such that when the contamination shield is aligned with the substrate, those holes align with transfer agent layer positions.

In certain embodiments, contamination shield layer holes may be shaped such that they allow genetic material to pass through the contamination shield during a transfer of genetic material to at least one target compartment when the contamination shield is aligned with the substrate and target compartments. In certain of these embodiments, the contamination shield layer may be positioned and shaped such that the contamination shield layer helps to minimize the transfer of genetic material into target compartments that are not aligned with the particular genetic material position or transfer agent space.

In certain embodiments, a contamination shield layer may comprise a plastic material such as, but not limited to, acrylate and PMMA. In certain embodiments, a contamination shield layer comprises a metal material such as, but not limited to, aluminum and/or steel. In certain embodiments, a contamination shield layer may be disposable or washable.

In certain embodiments, a sealing layer may be attached to a substrate. In certain embodiments, a sealing layer seals material into or onto a substrate. In certain embodiments, a sealing layer seals genetic material into or onto a substrate such that the genetic material is physically contained in or on the substrate.

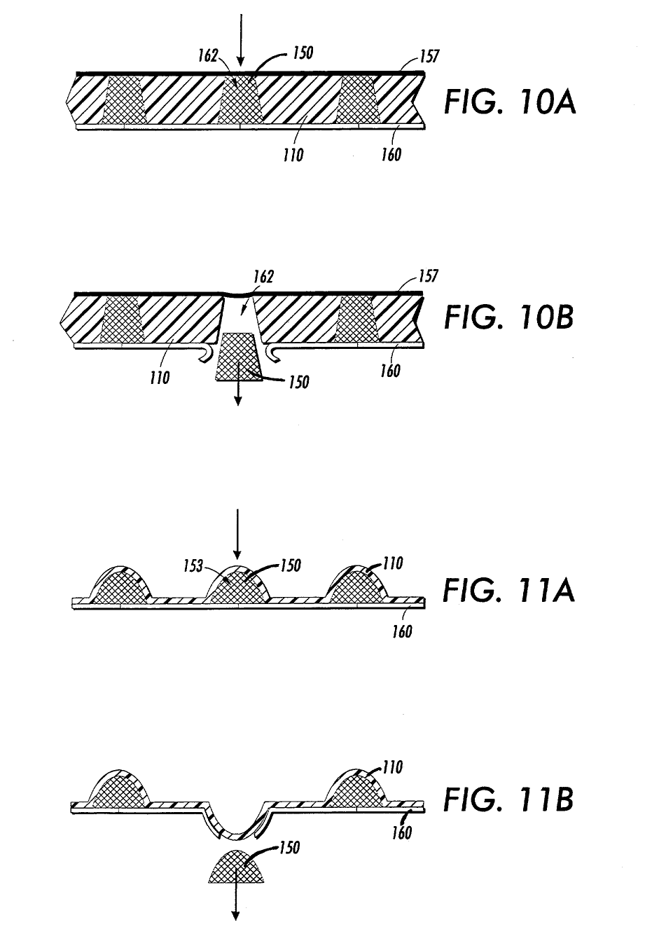

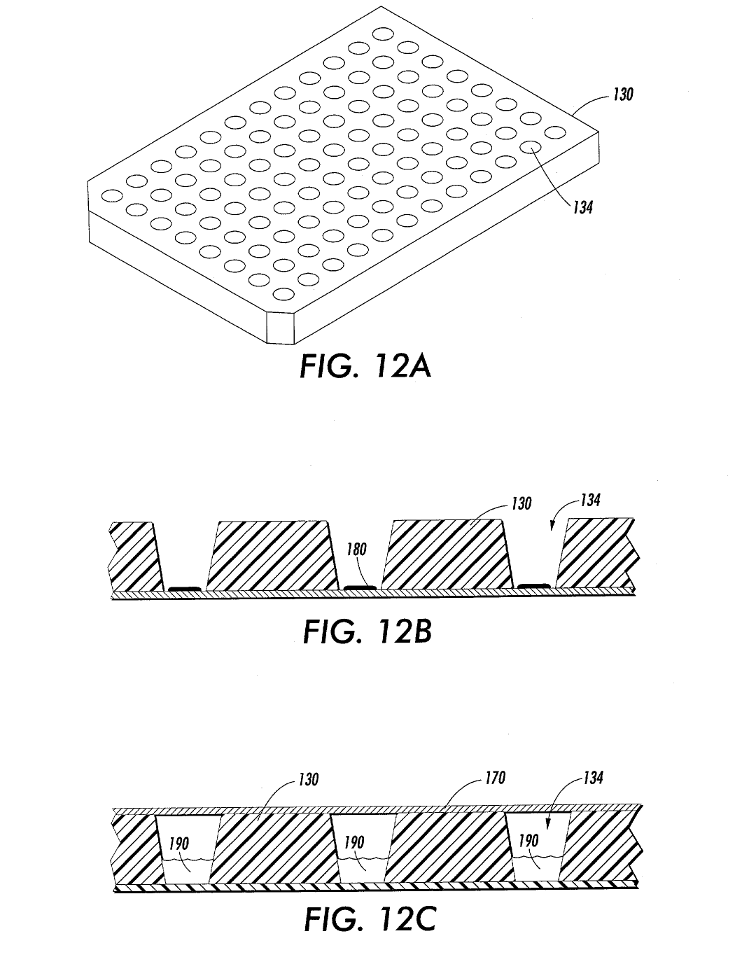

In certain embodiments, a sealing layer comprises a contiguous layer, wherein at least a majority of the sealing layer does not contain holes. In certain embodiments, a sealing layer contains no holes. In certain of these embodiments, the sealing layer may be made of a material such that a transfer agent layer may be forced through the sealing layer by pressure from a pressure source (see, e.g., FIGS. 10 and 11). In certain embodiments, the sealing layer is a thin material that allows a transfer agent layer to be forced through the sealing layer. In certain embodiments, a sealing layer may be pre-slitted such that it facilitates the rupture of the sealing layer by the transfer of a transfer agent layer.

In certain embodiments, a sealing layer may be attached to a substrate. In certain embodiments, a sealing layer is attached to a substrate such that the sealing layer stays in place during storage and distribution. In certain embodiments, a sealing layer may be attached to a substrate at multiple points such that separate regions of the sealing layer may be individually ruptured independently of the sealing layer in other regions.

In certain embodiments, the sealing layer may be attached to a multiwell plate such that two or more of the wells of the multiwell plate are sealed by the sealing layer. In certain embodiments, the sealing layer may be attached to a multiwell plate at multiple points such that two or more wells of the multiwell plate are sealed independently of each other. In certain of these embodiments, the sealing layer may be ruptured at the position of a well independently of the sealing layer at the positions of other wells.

In certain embodiments, a sealing layer may comprise materials such as, but not limited to, Mylar, Kapton, or plastic wrap. In certain embodiments, a sealing layer may be attached to a substrate by an adhesive. In certain embodiments, the adhesive is a double-side dry-film adhesive. In certain embodiments, the adhesive is 10 micrometers thick or less. In certain embodiment, the holes in the adhesive are the same size or are larger than the transfer agent spaces in the matrix. In certain embodiments, the holes in the are smaller than the transfer agent spaces in the matrix.

In certain embodiments, a protective foil is laminated onto the adhesive. In certain embodiments, the protective foil is laminated by low-temperature thermal plus pressure lamination. In certain embodiments, the protective foil has preformed slits at one or more positions that will align with genetic material positions when the foil is laminated onto the adhesive. In certain of these embodiments, the preformed slits have two flaps that overlap with each other.

In certain embodiments, a target compartment may contain a solution at the time a transfer agent layer is transferred into the target compartment. In certain embodiments, the target compartment may be empty at the time a transfer agent layer is transferred into the target compartment. In certain embodiments, a target compartment may contain dried reaction reagents such as, but not limited to, PCR reagents, at the time a transfer agent layer is transferred into the target compartment.

In certain embodiments, a transfer agent layer that is transferred into a target compartment dissolves after contact or immersion in a solution contained in the target compartment. In certain embodiments, a transfer agent layer that is transferred into a target compartment may be dissolved after contact or immersion in a solution contained in the target compartment with the application of agitation or heat.

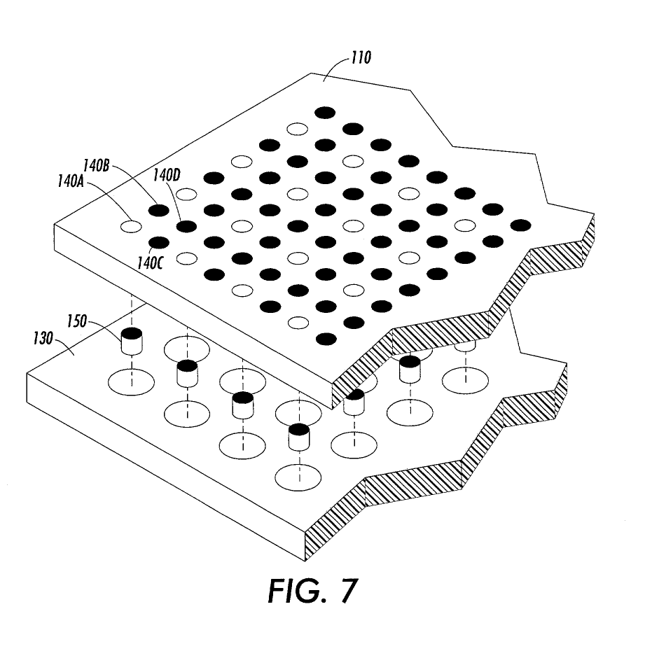

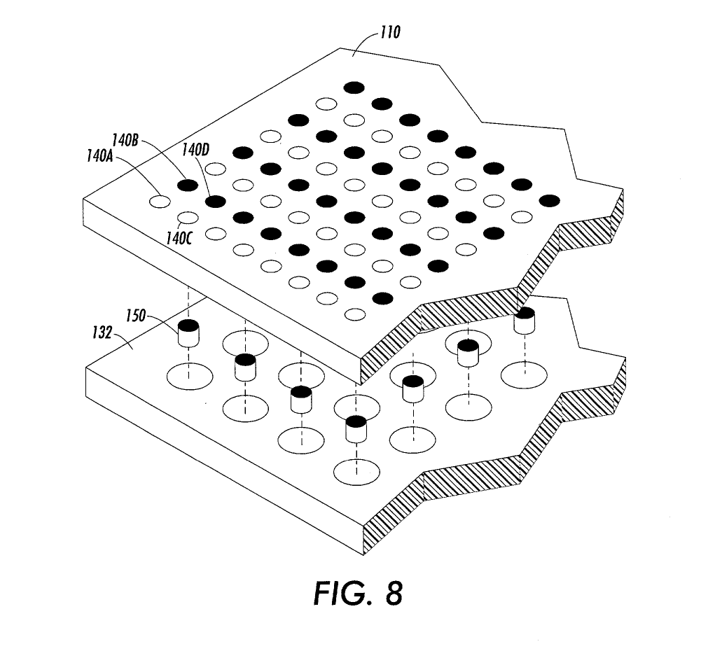

In certain embodiments, multiple transfer agent layers may be released into multiple plates by transferring the transfer agent layers from only a portion of the transfer agent spaces for each plate. For example, in certain embodiments, the transfer agent layer of the first of every four transfer agent spaces in a matrix that includes 384 transfer agent spaces is released into a first 96-well plate (see e.g. FIGS. 7 and 8). For the next 96-well plate, the transfer agent layer of the second of every four transfer agent spaces is transferred and so on. In this way, in certain embodiments, the 384 transfer agent spaces can be used to transfer genetic material to four separate 96-well plates (see, e.g., FIGS. 7 and 8).

In certain embodiments, a substrate comprises a multiwell plate cover comprising protrusions with capillaries within them. In certain embodiments, at least two of the protrusions contain a solution of genetic material from at least two different individuals in the capillary of the protrusion. In certain embodiments, genetic material in a capillary may be transferred to a well of a multiwell plate. In certain embodiments, the transfer occurs with centrifugation of the plate with the cover seated on top of the plate. In certain embodiments, genetic material in a capillary may be transferred by the inherent reflux that occurs during the high temperature part of a PCR cycle to a well of a multiwell plate containing the liquid phase of a PCR reaction.

In certain embodiments, a cover with protrusions is removed after transfer of genetic material to one or more wells of a multiwell plate and is replaced with a cover without protrusions. In certain embodiments, a replacement cover for a multiwell plate may be constructed of material that permits the transmission of light, such as UV light, sufficient to allow the transmission of sufficient excitation light to produce detectable light emission. In certain embodiments, light with a wavelength of at least 250 nm may be transmitted through the replacement cover. In certain embodiments, the optically transparent areas will not constitute the entire cover, but may be located above at least a majority of the wells where genetic material will be analyzed. In certain embodiments, the optically transparent areas may be located above at least two wells of a multiwell plate. In certain embodiments, these optically transparent areas are large enough to allow the transmission and collection of sufficient light signal for successful detection of the light signal.

Exemplary Detection Reactions

In certain embodiments, the presence or absence of defined genetic sequences in the deposited genetic material is detected. In various embodiments, there are various methods that can be employed to generate a signal that correlates with the presence or absence of the defined genetic sequence.

In certain embodiments, the presence of certain genetic sequences can be detected by the application of an amplification reaction. Amplification reactions include, but are not limited to, PCR, RCAT, Ligase Chain Reaction, and Strand Displacement Amplification.

In a PCR reaction according to certain embodiments, one exposes the deposited genetic material to reaction reagents and to repeated cycles of different temperatures in order to generate multiple copies of a particular genetic sequence. In certain embodiments, the reaction reagents include, but are not limited to, polymerases, nucleotides, and primers. In certain embodiments, a set of primers comprises a first primer comprising a specific nucleic acid sequence that hybridizes to a predetermined target nucleic acid sequence in the deposited genetic material. The presence of the polymerase results in the addition of nucleotides to the 3' end of the first primer, and the nucleotides are typically added in a sequence specific manner depending on the sequence of the target genetic sequence to generate an amplified sequence complementary to the target. The temperature is changed such that the complementary amplified sequence and the target sequence denature. The conditions are then cycled for alternating rounds of amplification and denaturing.

In certain embodiments, the primer set includes a second primer comprising a specific nucleic acid sequence that hybridizes to the complement of the predetermined target nucleic acid sequence. The presence of the polymerase results in the addition of nucleotides to the 3' end of the second primer, and the nucleotides are typically added in a sequence specific manner depending on the complementary sequence of the target genetic sequence to generate an amplified sequence having the sequence of the target. The temperature is changed such that the amplified sequence and the target sequence denature. The conditions are then cycled for alternating rounds of amplification and denaturing.

In certain embodiments, one may design PCR reaction conditions such as temperatures, number of cycles, salt concentrations and primer design, to be suitable for particular sequences to be amplified. PCR is a well-established laboratory technology (Sambrook and Russell, Molecular Cloning: A Laboratory Manual, 3^{rd} Edition, Chapter 8, hereby expressly incorporated by reference in its entirety for any purpose), and it is routine for one skilled in the art to determine suitable PCR reaction conditions for amplification of any given genetic sequence or sequences without undue experimentation.

In certain embodiments, one can detect amplification sequences using a label. Various embodiments can employ any of many types of labels that can be detected including, but not limited to, fluorescent light, optical absorption, and radioactivity.

In certain embodiments, the invention provides for the fluorescent detection of the presence or absence of a defined genetic sequence. Fluorescent dyes that can be used include, but are not limited to, SYBR Green I, PicoGreen, and TOTO-1 (see Molecular Probes Handbook, section 8.3).

In certain embodiments, the invention provides for detection of the presence or absence of a defined genetic sequence through use of absorption dyes. In certain embodiments, the invention provides for detection of the presence or absence of a defined genetic sequence through use of Ethidium Bromide.

In certain embodiments, enzyme-based chromogenic detection and/or signal amplification may be used.

In certain embodiments, PCR may be used to detect variations in genetic sequences, for example, a single nucleotide polymorphism, different alleles, a deletion from a particular sequence, or a mutation. In certain embodiments, PCR may be used to differentiate between individuals that are homozygous (they have the same target sequence in both chromosomes) or are heterozygous (they have variation in the target sequence in each chromosome).

In certain embodiments, one can detect variation at a particular nucleotide position of a target nucleic acid sequence by using at least one primer that has a specific nucleotide at its 3' end that is complementary to the nucleotide to be detected at the particular nucleotide position.

In certain embodiments, one may detect two different target sequences that differ by the particular nucleotide at a particular position by using two primers. In certain embodiments, one of the primers has a specific nucleotide at its 3' end that is complementary to one of nucleotides of one of the two different target sequences. The other primer has a specific nucleotide at its 3' end that is complementary to the other of the nucleotides at the particular position of the other target sequence.

In certain of these embodiments, the two different primers can each be tagged with a different detectable label, including but not limited to, a fluorescent label. In certain embodiments, the fluorescent labels are red and blue. In certain of these embodiments, one may use a third primer that has the same sequence as a downstream region of the target sequence. The third primer will prime an extension reaction by hybridizing to the sequence that is complementary to the target sequence to provide for two way amplification.

In certain of these embodiments, the presence of a homozygous gene will be determined by detecting either pure blue or pure red, and the presence of a heterozygous gene will be determined by detecting both blue and red. The number of primers that can be designed is not limited. Thus, one can detect any number of nucleotides at a given position as well as any number of different sequence locations within the genetic material. In certain

What is claimed is:

1. A device comprising:
a) a substrate; and
b) a first genetic material position and a second genetic material position on the substrate that each comprise genetic material attached to the substrate, wherein the genetic material of the first genetic material position comprises purified genomic DNA comprising more than one chromosome of the genomic DNA from at least one cell of an individual within a defined population and the genetic material of the second genetic material position comprises purified genomic DNA comprising more than one chromosome of the genomic DNA from at least one cell of an individual within a defined population, and wherein the genetic material of the first genetic material position is separate from the genetic material of the second genetic material position.

2. The device of claim 1, wherein the first genetic material position comprises purified genomic DNA comprising more than ten chromosomes of the genomic DNA from at least one cell of an individual within a defined population and wherein the second genetic material position comprises purified genomic DNA comprising more than ten chromosomes of the genomic DNA from at least one cell of an individual within a defined population.

3. The device of claim 1, wherein the first genetic material position comprises purified genomic DNA comprising more than 80% of the genomic DNA from at least one cell of an individual within a defined population and wherein the second genetic material position comprises purified genomic DNA comprising more than 80% of the genomic DNA from at least one cell of an individual within a defined population.

4. The device of claim 1, further comprising additional genetic material positions on the substrate that each comprise genetic material attached to the substrate and wherein the genetic material of each of a majority of the additional genetic material positions are separate from one another.

5. The device of claim 1, further comprising 94 additional genetic material positions on the substrate that each comprise genetic material attached to the substrate and wherein the genetic material of each of a majority of the additional genetic material positions are separate from one another.

6. The device of claim 5, wherein the genetic material of each of a majority of the genetic material positions derives from a different individual of the defined population.

7. The device of claim 1, further comprising 382 additional genetic material positions on the substrate that each comprise genetic material attached to the substrate and wherein the genetic material of each of a majority of the additional genetic material positions are separate from one another.

8. The device of claim 1, wherein the substrate comprises paper.

9. The device of claim 1, wherein the substrate comprises a cover.

10. The device of claim 9, wherein the cover comprises a cover for a multiwell plate, and wherein the cover comprises at least two protrusions, wherein each of the at least two protrusions extend into separate wells of the multiwell plate when the cover is placed on the multiwell plate.

11. The device of claim 10, wherein each of the at least two of the protrusions comprises a capillary.

12. The device of claim 5, wherein the substrate comprises a multiwell plate.

13. The device of claim 12, wherein the multiwell plate is a 96-well plate.

14. A method for distributing genetic material, comprising distributing at least one device to at least one recipient; wherein the device comprises a substrate and a first genetic material position and a second genetic material position on the substrate that each comprise genetic material deposited onto the substrate, wherein the genetic material of the first genetic material position comprises purified genomic DNA comprising more than one chromosome of the genomic DNA from at least one cell of an individual within a defined population and the genetic material of the second genetic material position comprises purified genomic DNA comprising more than one chromosome of the genomic DNA from at least one cell of an individual within a defined population, and wherein the genetic material of the first genetic material position is separate from the genetic material of the second genetic material position.

15. The method of claim 14, wherein the devices are distributed to at least ten recipients.

16. The method of claim 14, wherein the substrate comprises a multiwell plate.

17. The method of claim 16, wherein the multiwell plate is one of a 96-well plate and a 384-well plate.

18. The method of claim 17, wherein each of a majority of the genetic material positions on the substrate comprises a solution comprising the genetic material.

19. The method of claim 16, wherein each of a majority of the wells of the multiwell plate contain a separate genetic material position.

20. The method of claim 14, wherein the at least one device is distributed with information regarding the genetic material deposited on the substrate.

21. The method of claim 20, wherein the information comprises genotyping information correlated with at least one position of the deposited genetic material.

22. The method of claim 21, wherein the genotyping information comprises information identifying at least one of an allele, mutation, single nucleotide polymorphism and nucleotide sequence deletion in the deposited genetic material.

23. A device comprising
a) a substrate comprising
1) a matrix;
2) one or more separate transfer agent spaces; and
3) one or more transfer agent layers contained in one or more of the separate transfer agent spaces; and
b) genetic material deposited on or in one or more of the transfer agent layers.

24. The substrate of claim 23, wherein at least one of the transfer agent layers comprises a saccharide selected from at least one of sucrose and glucose.

25. The substrate of claim 23, wherein the transfer agent layers are contained in at least 96 separate transfer agent spaces.

26. The substrate of claim 25, wherein each of a majority of the at least 96 transfer agent spaces is alignable with a well of a 96-well plate.

27. The substrate of claim 23, wherein the transfer agent layers are contained in at least 384 separate transfer agent spaces.

28. The substrate of claim 27, wherein each of a majority of the at least 384 transfer agent spaces is alignable with a well of a 384-well plate.

29. The substrate of claim 26 wherein the genetic material on each of a majority of the transfer agent layers comprises purified genomic DNA comprising more than one chromosome of the genomic DNA from at least one cell of an individual within a defined population.

30. The substrate of claim 28 wherein the genetic material on each of a majority of the transfer agent layers comprises purified genomic DNA comprising more than one chromosome of the genomic DNA from at least one cell of an individual within a defined population.

31. The substrate of claim 29, wherein each of a majority of transfer agent layers contains genetic material from a different individual of a defined population.

32. The substrate of claim 30, wherein each of a majority of transfer agent layers contains genetic material from a different individual of a defined population.

33. The device of claim 23, further comprising a barrier layer positioned on one side of the substrate.

34. The device of claim 23, further comprising a sealing layer positioned on one side of the substrate.

35. A method for distributing genetic material, comprising distributing at least one device to at least one recipient; wherein the device comprises a substrate comprising a matrix; one or more separate transfer agent spaces that extend through or partially through the matrix; one or more transfer agent layers contained in one or more of the transfer agent spaces; and genetic material deposited on or in one or more of the transfer agent layers.

36. The method of claim 35, wherein the devices are distributed to at least ten recipients.

37. The method of claim 35, wherein the at least one device is distributed with information regarding the genetic material contained on the substrate.

38. The method of claim 37, wherein the information comprises genotyping information correlated with at least one position of the deposited genetic material.

39. The method of claim 38, wherein the genotyping information comprises information identifying at least one of an allele, mutation, SNP and nucleotide sequence deletion in the deposited genetic material.

40. A device comprising:
a) a substrate;
b) a transfer agent layer; and
c) a first genetic material position and a second genetic material position on or in the transfer agent layer that each comprise genetic material, wherein the genetic material of the first genetic material position comprises purified genomic DNA comprising more than one chromosome of the genomic DNA from at least one cell of an individual within a defined population and the genetic material of the second genetic material position comprises purified genomic DNA comprising more than one chromosome of the genomic DNA from at least one cell of an individual within a defined population, and wherein the genetic material of the first genetic material position is separate from the genetic material of the second genetic material position.


SEQUENCE LISTING (5 sequences)

SEQ ID NO: 1 - DNA, 18 bp, Artificial Sequence
  Description of Artificial Sequence Artificial DNA primer
tataaatagggcctcgtg

SEQ ID NO: 2 - DNA, 25 bp, Artificial Sequence
  Description of Artificial Sequence Artificial DNA primer
cagggtgctgtccacactggctcgc

SEQ ID NO: 3 - DNA, 20 bp, Artificial Sequence
  Description of Artificial Sequence Artificial DNA primer
ctcgttcaccactttcttgc

SEQ ID NO: 4 - DNA, 20 bp, Artificial Sequence
  Description of Artificial Sequence Artificial DNA primer
gggaagctgggttggggggc

SEQ ID NO: 5 - DNA, 15 bp, Artificial Sequence
  Description of Artificial Sequence Artificial DNA primer
ggtgctgtccacact